(19) United States
(12) Patent Application Publication
(10) Pub. No.: US 2021/0259985 A1
(43) Pub. Date: Aug. 26, 2021

(54) METHOD OF PROVIDING CBD OIL WITHIN POLYMER FIBROUS STRUCTURES FOR SUBSEQUENT TRANSFER TO A USER'S SKIN

(71) Applicant: WeDoWee, LLC, Middletown, DE (US)

(72) Inventor: David HALL, Auburn, AL (US)

(21) Appl. No.: 17/176,260

(22) Filed: Feb. 16, 2021

Related U.S. Application Data
(60) Provisional application No. 62/978,948, filed on Feb. 20, 2020.

(51) Int. Cl. A61K 9/70; A61K 31/05; A61K 47/34; D06P 3/82; D06M 13/152; A61K 9/00

(52) U.S. Cl. CPC A61K 9/70; A61K 31/05; A61K 47/34; D06M 2101/32; D06M 13/152; A61K 9/0014; D06P 3/8214

(57) ABSTRACT

A method of infusing CBD oil into polyester yarns or polymer fibrous structures to establish reservoirs of CBD in said yarns or structures of sufficient integrity to withstand multiple washing cycles while periodically allowing migration of CBD from said reservoirs to the skin of a wearer of a product made from the infused yarns or polymer fibrous structures

### BACKGROUND OF THE INVENTION

Cannabidiol (CBD) has been long promoted as a beneficial distillate of hemp (cannabis) for use in alleviating many painful conditions.

There are eight major cannabinoids produced by the cannabis plant. CBD is one of the most prevalent active ingredients of cannabis. CBD is claimed to deliver an option for treating different types of chronic pain. It is known that CBD applied on the skin has helped lower pain and inflammation caused by arthritis and plantar fasciitis. One study has demonstrated the mechanism by which CBD inhibits inflammatory and neuropathic pain, two of the most difficult types of chronic pain to treat. In view of recent changes in the law, CBD distillate is typically combined with a carrier oil, grapeseed oil or hemp oil, to form CBD oil is now available from cannabis processors.

Aside from applying CBD oil directly to the skin for relief, it has been proposed to apply the CBD oil to fabric and/or clothing for release to the skin of a wearer to deliver relief.

### SUMMARY OF THE PRESENT INVENTION

The present invention provides a new and improved method for infusing a carefully selected CBD oil into fibers in the early stages of fabric and clothing manufacture. The CBD oil infused into the fibers establishes a reservoir of CBD which eventually migrates to the skin of the wearer of fabric or clothing made from the CBD infused fibers. CBD treated socks have been proposed since aching feet are one of the common sources of chronic pain. The CBD infused fibers in the user's sock will be a source for pain relief during sleep or daytime wear.

Specially designed socks or compression joint sleeves that can achieve both high adsorption of the oil and the ability to transfer the oil to the user can be achieved, in accordance with the invention, by employing two specifically designed textile polymer types that have been found to be the most suitable for maximum oil adsorption. These polymers are nylon and polyester which have specific characteristics that are superior for socks and compression sleeves. It is understood that the specially designed fiber types described herein have application to other end uses besides socks and body part support compression sleeves, e.g., wraps or apparel coverage for other areas of the body.

Not all wearable fibers such as cotton, rayon, wool and comparable other fibers are suitable for the application of the CBD oil, but the developed socks or sleeves may contain small amounts of these fibers or blends thereof which act to absorb body moisture and add comfort. The primary microfibers of rayon (also marketed under “Bamboo,” “Tencel” and other trademark labels), because of their increased surface area, are ideal for practice of this invention. Tencel is the brand name of a rayon lyocell.

### DETAILED DESCRIPTION OF THE INVENTION

In accordance with the invention, a fundamental key to the improvement in adsorption and desorption of the CBD oil is the use of microfibers. For example, a fiber with a diameter of 2 micron has a surface of 2 pi (C=pi D). Four fibers having a diameter of 1 micron will have the same weight as the 2 micron fiber but will have 4 pi surface area, i.e., double the surface area of the 2 micron fiber. This means that 100% more CBD oil infused fibers will be in contact with the user's skin.

Another key factor that can measurably increase the surface area of both polyester and nylon fibers is the use of fibers spun in a trilobal or a round shape. Nylon has the advantage over polyester in that it can be treated with the oil at a much lower temperature. Polyester (100%) with a crystalline fiber structure is normally dyed at elevated temperature which can only be achieved in a dye bath employing high pressure and small dye molecules (disperse dyes) in the dyeing process. They must also use a class of dyeing assistant molecules called carriers which help to wedge the chain structures apart to allow the small disperse dye molecules to enter the fibers.

The problem of ineffective CBD oil penetration into 100% polyester is solved by copolymerizing the polyester with nylon type molecules. This fiber blend combination (polyester/polyamide) greatly allows the resulting copolymer to be significantly more easily dyed at temperatures well below the boil point while also allowing greater penetration and adsorption of the CBD oil. This also allows for better desorption of the CBD oil onto the user's skin. This combination of microfibers in a round and/or especially in a trilobal shape will greatly facilitate the use of CBD oil in combination with these and other polyesters having cross sectional shape.

### Example 1

The principles of the invention are manifested in the following package dyeing example, wherein CBD oil is infused into a yarn made from a blend of polyester or polyamide polymer such as (75% Nylon-25% Tencel [a Rayon] by weight) fibers which are trilobal or round in cross-section.

The yarn is initially produced from blending the polyester and polyamide fibers (trilobal or round). The blend is then fed into a carding machine to produce a sliver which is then drawn down to a size suitable for spinning.

The fiber blend is then spun into yarn and wound onto spools. Subsequently, the spools are package dyed under pressure with an appropriate dyestuff (black for example) and are immediately subsequently infused with CBD oil which further serves as a lubricant in the process. The colored/CBD infused yarn now having “micro-reservoirs” of CBD in each strand is dried at 220-225° F. to bind the CBD to the yarn fibers and the rewound spools of dyed/CBD infused yarn are then converted through conventional weaving or knitting processes into fabrics or products such as sleeves or socks, the conversion combining the individual yarn “micro-reservoirs” into “macro-reservoirs” of CBD in a textile fabric product.

In accordance with the invention, the sleeves or socks or other converted products have CBD “macro-reservoirs” formed from the hemp oil in the individual strand “micro-reservoir” copolymer fiber substrates which will withstand multiple laundering cycles. There will be enough of the oil available at the fiber surfaces to migrate to the skin of a suffering wearer, thereby providing significant CBD exposure and concomitant reduction of pain for at least 35 launderings. The converted products contain textile fibers that have high substantivity so they absorb enough of the oil to act as a “macro-reservoir” of the oil so that an effective amount of pain-relieving CBD migrates to the fiber surfaces and into contact with the skin of the wearer after at least 35 launderings.

The “micro-reservoirs” have resistance to being destroyed during laundering. Thus, only hydrophobic fibers are suitable for use with this method. For example, cellulosic fibers are not useful primary choices since absorbed oil can be emulsified with the detergents and more easily removed, although such fibers may be blended with polyester fibers to provide a degree of comfort. The cellulosic Rayon fiber (Tercek is useful for it moisture adsorption. The skin of this fiber helps to prevent the loss of CBD the during laundering,

In order to maximize the penetration and adsorption of the CBD oil, it is applied as the last step of the package dyeing process. This dyeing technique allows increasing the dyeing pressures causing the dyebath temperature to be several (5 to 8) degrees above the boiling point of water. This will allow for greater penetration of the CBD resulting in a higher CBD reservoir content of the fibers.

More specifically, the practice of the invention involves control of important variables required for the most effective penetration and durability of CBD oil into yarns, fabrics and compressed materials that can be used in products to effectively deliver substantial therapeutic effect for the end user. The specific variables are:

### (1) Fiber Properties

The fibers are chosen to be ideally nylon or polyester or other polymer polymerized from oil monomers to ensure the retention of the CBD oil in the fiber for a minimum of 35 laundry cycles. Nylon or polyester among other oil base fibers can be blended with other fibers. Although not essential, trilobal shaped fibers would be most ideal because they increase the amount of surface area available for the CBD oil to be in contact with the skin.

### (2) Process Temperature

A temperature of 225 degrees Fahrenheit is required, which allows the CBD oil molecules to become more substantive to the fiber polymer and to be retained within the fiber molecular structure (to the extent that it can endure a minimum of 35 laundry cycles), and still maintain a 50% level of CBD oil presence within the fibers. This allows for the continuous migration of the CBD oil from the surface of the fibers to the skin surface, thereby allowing for absorption into the body of the end user. The critical required temperature can be achieved using various techniques known to the textile industry. These include a pressurized dyeing process which would include package yarn dyeing, or jet or beam dyeing of woven or knit fabrics. In addition, this temperature can be achieved in the manufacturing of compressed materials in which the CBD Oil is applied by a spray method, or liquid bath application in which the material is subject to temperatures of 225 degrees Fahrenheit in the drying process using a heated chamber or heated rollers.

### (3) Purity of CBD

The purity level of the CBD oil must be a minimum of 20% in order to achieve the most therapeutic benefit for the end user.

The CBD oil used in the practice of the invention is ideally a full spectrum organically grown CBD oil produced by distillation with cold press extraction. This maintains the useful cannabinoids for topical application but does not convert the THC-A to THC in significant amounts (trace levels only—less than 0.03 percent) and maintains useful terpenes without employing solvents.

The CBD oil employed is specifically described as follows:

CBD OIL FOR INFUSION

| Component | Weight % | Concentration | |
|---|---|---|---|
| THC-A | -0- | -0- | |
| D9 − THC | .10 | .98 | mg/ml |
| CBD | 2.40 | 23.14 | mg/ml |
| CBG | .04 | .36 | mg/ml |
| CBC | .09 | .90 | mg/ml |
| CBDA | .15 | 1.41 | mg/ml |
| TOTAL | 2.78 wt % | 26.79 | mg/ml |
| MAX THC | .10 wt % | .98 | mg/ml |
| MAX CBD | 2.53 wt % | 24.37 | mg/ml |

RATIO of TOTAL CBD to THC 24.8:1

The carrier oil for the full spectrum CBD is hemp seed oil and/or grapeseed oil (long chain free fatty acid oils) which contribute to effective phytocannabinoid transport via the hepatic and/or lymphatic pathways of the end user.

In the batch dyeing example of the invention, the polyester yarns are wound on cones which are subjected to a standard dye both to impart color to the yarns and, immediately thereafter, are subjected to a bath of the CBD oil at 220-225° F.

The ability to increase pressure in some specialized dyeing machines allows the temperature to increase above the boiling point. At 220-225° F. the pores of the fiber open up to a greater degree which allows CBD molecules to penetrate into the fiber molecules. When the dyes and CBD oil penetrate deep into the fiber, it is more resistant to washing out. This would not be the case when the molecules are on the surface of the fibers.

Package dyeing machines are used for yarns as in the exemplary method. It is contemplated that Beam Dyeing machines can be used for woven fabric and Jet Dyeing machinery for knit fabrics. For compressed material (non-woven and non-knit), the machinery application of CBD oil may be performed using a spray method or passage through a liquid bath which includes the CBD oil. In all these processes, the CBD oil is to be subjected to 220-225° F. heat by passage through a heated chamber or heated rollers that are temperature controlled using either steam or electricity in a non-pressurized environment.

In the practice of the invention, the weight ration of CBD oil to yarn and/or fabric and/or non-woven material is five pounds of CBD oil to 100 pounds of yarn/fabric/material, i.e., 5% of the weight of the goods.

The dye bath temperature of 220-225° F. is for pressurized equipment which speeds up the process and is used for both the dye and the CBD infusion.

The infusion of CBD oil into polyester/polyamide copolymer yarns of 75% nylon and 25% Tencel by weight in the above-described batch dyeing process results in an infusion of CBD oil which readily withstands 35 laundering cycles while retaining sufficient CBD in the formed yarn reservoir to effectively migrate to the skin of the end user.

It should be understood, of course, that the specific form of the invention herein illustrated and described is intended to be representative only, as certain changes may be made therein without departing from the clear teachings of the disclosure. Accordingly, reference should be made to the following appended claims in determining the full scope of the invention.

1. The method of establishing a dynamic micro reservoir of CBD analgesic in a yarn strand convertible to a fabric in which multiples of the yarn micro reservoirs combine to form a fabric macro reservoir of CBD which fabric is convertible to an article of clothing adapted to permit migration of CBD from the macro reservoir to the skin of a wearer of the article of clothing to provide beneficial analgesic effects, comprising the steps of:
a) selecting a polyester microfiber yarn;
b) forming the yarn into a treatment spool;
c) subjecting said spool of yarn to a dye bath maintained at 220-225° F.;
d) subsequently subjecting said spool to a bath of full-spectrum CBD oil maintained at 220-225° F., said CBD oil having a composition including substantial amounts of CBD and trace amounts of not more than 0.03% THC and said CBD being infused into said yarn in a ratio of 5/100 by weight; and
e) said macro reservoir having sufficient integrity to withstand multiple laundering cycles.

2. The method of claim 1 in which the CBD oil has a purity of at least 20%.

3. The method of claim 1 in which the micro-fiber is 75% Nylon—25% Tencel by weight.

4. Method of infusing CBD into a fibrous material comprising:
a) selecting the fibrous material from a group including polyester micro fiber yarns; woven polyester micro fiber fabric; non woven polyester micro fiber fabric; knitted polyester fabric;
b) applying full spectrum CBD oil with at least 20% purity to the fibrous material at temperatures at least 220-225 degrees F. in a weight ratio of 5% of applied CBD to fibrous material;
c) whereby said infused fibrous material has a reservoir of CBD of sufficient integrity to withstand multiple laundering cycles.
